(19) United States
(12) Patent Application Publication
(10) Pub. No.: US 2008/0097534 A1
(43) Pub. Date: Apr. 24, 2008

(54) Resuscitation system

(76) Inventors: Helge Myklebust, Stavanger (NO); Oystein Stromsnes, Tau (NO)

Correspondence Address: DORSEY & WHITNEY LLP;INTELLECTUAL PROPERTY DEPARTMENT, SUITE 3400, 1420 FIFTH AVENUE, SEATTLE, WA, 98101, US

(21) Appl. No.: 11/584,257

(22) Filed: Oct. 20, 2006

(51) Int. Cl. A61N 1/39

(52) U.S. Cl. 607/5

(57) ABSTRACT

A resuscitation system includes a chest compression device to repeatedly compress the chest of a patient and thereafter cause or allow the chest to expand. The resuscitation system also includes a defibrillator to apply electric impulses to the heart, a measuring device for measuring at least one characteristics of the resuscitation process, and a signal processor for controlling operation of the chest compression device and/or the defibrillator. The defibrillator may be an integrated or external device working in a master/slave relationship with the remaining system.

### TECHNICAL FIELD

This invention is related to resuscitation systems, and, more particularly, to a resuscitation system for providing CPR and defibrillation.

### BACKGROUND OF THE INVENTION

Sudden cardiac arrest is a leading cause of death in developed countries in the Western World, like United States and Canada. To increase the chance for survival from cardiac arrest, Cardio Pulmonary Resuscitation (“CPR”) and heart defibrillation should be given in the first few critical minutes after the incident. CPR is performed to ensure a sufficient flow of oxygenated blood to vital organs by external compression of the chest combined with rescue breathing. Heart defibrillation is performed to re-establish normal heart rhythm by delivery of an external electric shock.

The quality of CPR is an important factor in survival rate. To maximize the chances for survival, chest compressions must be given with a minimum of interruptions, and be of sufficient depth and rate. Performing chest compressions manually is an extremely exhausting task, and it is practically impossible to give manual CPR of sufficient quality during transportation of a patient. A successful defibrillation depends on the metabolic state of the heart when the electrical shock is applied. Defibrillation must be performed at a determined times during the compression cycle. In general, defibrillators comprise an integrated (or external) electrocardiograph device (ECG) that records the electrical activity in the heart in a continuous manner and permits determination of the correct time for applying an electrical shock. Chest compressions cause noise in the ECG signal and can lead to incorrect determination of the time for defibrillation. For this reason, in most cases, chest compressions are performed on a patient either manually or automatically, and the process is stopped in order to get a correct ECG signal before applying defibrillation. This leads to relatively long periods without chest compression and to cumbersome procedures where different equipment must placed on the patient.

U.S. Patent Publication No. 2006/0155222 describes a device for performing chest compressions for CPR in coordination with applying electrostimulus for additional resuscitative actions, such as defibrillation. This publication describes only the timing of the defibrillator shock with respect to the CPR cycle, specifically near the end of a compression cycle.

U.S. Pat. No. 6,807,442 describes a system for reducing signal disturbances in ECG caused by cardio-pulmonary resuscitation (CPR). The system includes a measuring device for measuring one or more signals derived from parameters such as compression depth, lung inflation etc. as a result of CPR. The parameter signals form one or more reference signals that correlate with the signal disturbances. One or more adaptive filters filter the signals from the signal that constitutes the ECG signals so as to remove disturbances caused by CPR.

The systems described in these publications do not address the problem of providing a resuscitation system where signals representing characteristics of the resuscitation process are adapted for use in controlling the process.

### SUMMARY OF THE INVENTION

A resuscitation system according to the present invention comprises a chest compression device to repeatedly compress the chest of a patient and then cause or allow the chest to expand. The resuscitation system also includes a defibrillator to apply electric impulses to the heart, a measuring device for measuring characteristics of the resuscitation process, and a signal processor for controlling operation of the chest compression device and/or the defibrillator. The resuscitation system can comprise an integrated defibrillator or a docking station and/or connection(s) for receiving and/or interfacing an external defibrillator. The alternative using an external defibrillator permits use of a system according to the invention together with any defibrillators available in the market. The connection between the defibrillator and the remaining system may be wired or wireless. The defibrillator may have its own power supply or may share power supply with the resuscitation system.

The signal processor used in the resuscitation system can be adapted to control operation of the chest compression device and/or the defibrillator based on measured characteristics of the resuscitation process. The processor device can also be adapted to control operation of the chest compression device and/or the defibrillator based on predetermined characteristics of the resuscitation process, or it can be adapted to control operation of the chest compression device and/or the defibrillator based on comparison of measured and predetermined characteristics of the resuscitation process. The predetermined characteristics may for example be characteristics recommended in the international guidelines for resuscitation.

The signal processor may be integrated in the resuscitation system or may be a part of an external defibrillator. When connected together, the control operations may be performed by the defibrillator, the defibrillator being the “master” and the remaining system a “slave”, or the control operations may be performed by the system, the system being “master” and the defibrillator “slave”.

### BRIEF DESCRIPTION OF THE DRAWINGS

FIG. 1 is a block diagram of an embodiment of the invention.

FIG. 2 is an isometric view of an embodiment of the invention.

FIG. 3 is a graph showing an example of VF signals and other signals caused by CPR.

FIG. 4 is exemplary decision model showing an estimated ECG curve.

### DETAILED DESCRIPTION OF PREFERRED EMBODIMENTS

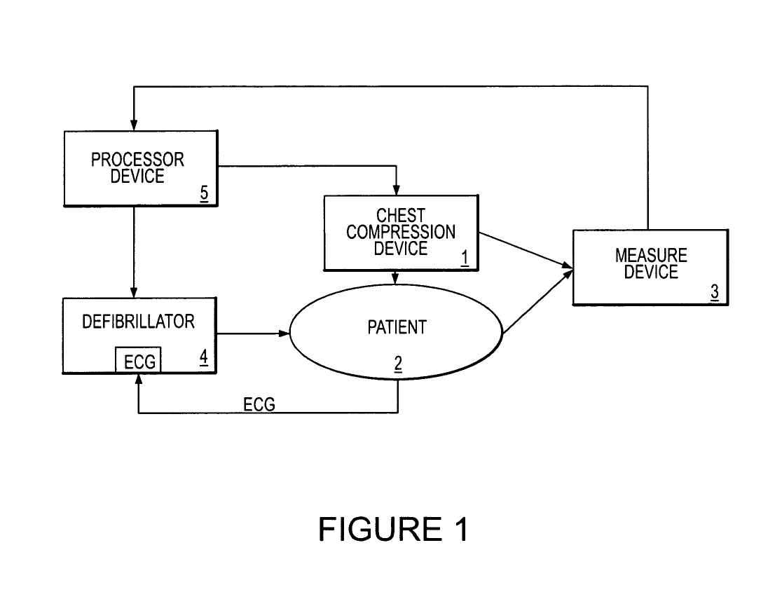

FIG. 1 is a block diagram of an embodiment of the resuscitation system according to the invention. This block diagram shows a chest compression device 1 for repeatedly compressing the chest of a patient 2 to cause or allow the chest to expand. The device also includes a measuring device 3 for measuring characteristics of the resuscitation process. The measuring device 3 may be implemented using any sensors or other measuring devices suitable for measuring characteristics of the resuscitation process, and/or other relevant information regarding CPR in the system and/or in the patient.

The embodiment of the resuscitation system shown in FIG. 1 also includes a defibrillator with an ECG device 4, and a signal processor 5 connected to the measuring devices 3 and/or the chest compression devices 1 and/or the defibrillator 4 to control the operation of the chest compression device 1. In this embodiment of the invention, the signal processor 5 may be connected to a data-storing device (not shown) to permit storage of measurement values and thus provide historical data. These stored values may later be used for evaluating the resuscitation episode. In this way systematic or occasional operator errors may also be revealed and this knowledge may be used to adjust procedures and/or train personnel. Stored values may also be used to reveal equipment errors and initiate service. The signal processor 5 may also connected to a display device (not shown) to display characteristics of the resuscitation process, and/or alarms. The signal processor 5 is adapted to receive signals from the measuring device 3 and the ECG in defibrillator 4, to filtrate chest compression noise, and, based on the filtered signal, to provide either an indication to a user of the correct time to defibrillate or a start signal to the defibrillator 4.

In operation, the signal processor 5 used in the resuscitation system can be adapted to control operation of the chest compression device 1 and/or the defibrillator 4 based on measured characteristics of the resuscitation process or based on a comparison of measured and predetermined characteristics of the resuscitation process. The predetermined characteristics may for example be characteristics recommended in the international guidelines for resuscitation. Present international guidelines describe the recommended time for activating defibrillation during CPR. After having performed the recommended period of time of CPR, for example, three minutes, the defibrillation is activated, the result of the defibrillation is analyzed, and CPR is continued for another period of time if the heart still has no rhythm. It is desirable to minimize the time intervals between CPR and defibrillation and between defibrillation and continued CPR, and this may be achieved by also using the signal processor for the defibrillator 4. For example may the resuscitation system according to the invention allow continuous CPR and activation of defibrillator during CPR. Other possible predetermined characteristics are a recommended number of compressions before defibrillation, presumed state of heart according to time from stop, characteristics of ECG such as “slope”, presence of VF, etc.

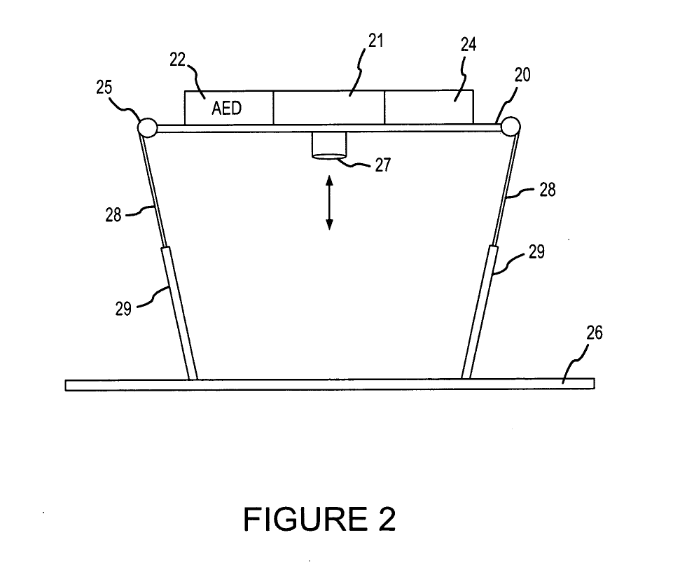

FIG. 2 is an isometric view of an embodiment of the invention. In this embodiment of the invention, a chest compression device 21, a signal processor (not shown in FIG. 2), a power supply (not shown) and a defibrillator 22 are mounted on a transverse plate 20. The signal processor 5 and the power supply are enclosed in a housing 24. The defibrillator 22 is in this illustration an automated external defibrillator (AED), which is integrated with the other elements of the system according to one embodiment of the invention. The AED 22 communicates seamless with the other elements/components of the system. The chest compression device 21 comprises in this embodiment a piston 27, a transmission mechanism (not shown) for transmitting energy to the piston 27 and a motor (not shown). The piston 27 moves in the direction of the arrow to perform compression and allow or cause decompression of the patient's chest.

The signal processor 5 comprises in this embodiment of the invention devices for controlling operation of the chest compression device 21 and the AED 22 based on predetermined characteristics and/or on characteristics measured by measuring devices (not shown). These measuring devices can be, for example, force sensors and/or depth sensors for measuring force/depth exerted/traveled by the compression device, compression counters, compression frequency counters, blood flow sensors for monitoring the blood flow of the patient, ventilation sensors for monitoring the ventilation flow, volume, and/or time interval of patient ventilation, impedance measuring means for measuring the impedance of the chest and thus give an indication of the ventilation of the patient, an electrocardiogram (ECG) device, tilt sensors for measuring the angle of the patient (whether the patient is lying, sitting/standing), position detectors for detecting the positioning and/or change of positioning of the chest compression device 21, battery power measurement means, internal motor temperature measuring means, etc. Control signals provided by the signal processor 5 may, for example, be based on patient characteristics, such as a measured chest height/depth of the patient, age of the patient, ECG measurements, etc.

The transverse plate 20 on which the above-described components are mounted is substantially rectangular and is connected on its short edges to two lateral legs, including an upper part 28 and a lower part 29. The connection between the transverse plate 20 and the upper part 28 of each of the legs is implemented by hinges 25 to permit to rotate the legs towards the transverse plate to provide a storage position for the resuscitation system. The upper part 28 is also situated telescopically inside the lower part 29 to permit easy step-less variation of the lengths of the legs. The legs are adapted for placement on the sides of the patient's body. The lower edge of the lower part 29 is connected to a back plate 26 adapted for placement under the patient's back.

The lower parts 29 of the legs can be fixed, shiftable, or rotatably connected to the back plate 26. In one embodiment of the invention, they are laterally shiftable in order to be able to be arranged in contact with the patient's body, and when in correct position they are fixedly connected to the back plate 26. In use, the chest compression device 21 is connected to the transverse plate 20 in such a way that the direction of the compression movement of the chest compression device 21 is substantially perpendicular to thorax in the area between the nipples. This may for example mean that the movement of the chest compression device 21 is substantially perpendicular to a plane comprising sternum, and substantially parallel to the back plate 18. The resuscitation system may be positioned relative to the patient's length by means of illustrations on the support or by other display devices that will be visible to an operator. In one embodiment the system may comprise physical devices or arrangements that indicate and/or guide the positioning of the system relative to the patient, for example, by arranging the legs so that they are placed in the patient's armpits, or by a rod indicating the distance to the patient's shoulders, etc.

During transport and storage, the legs may be separated from the back plate 26 thereby providing two separate sections, one section including the legs and the transverse plate 20 with the above mentioned devices on it, and the other one section including the back plate 26. The separate sections can be folded to a flat position to permit easy storage of the device. The support may be collapsible, demountable or foldable in order to minimize volume of the system when not in use. Preferably, the support is easy to assemble and prepare for use in order to minimize time wasted on assembling and mounting. This may for example be achieved by using spring-loaded elements which unfold themselves to the maximum size. The system should include as few separate parts as possible in order to minimize risk of incorrect assembly and to minimize assembly time. In other embodiments, the support can be a frame, stand, rack, tripod, etc. of suitable design.

The resuscitation system may also comprise ventilation devices (not shown). The ventilation devices may be regular ventilation devices, which may be operated by an operator, or they may be autonomous/automatic ventilation devices. If the ventilation devices are operated by an operator, the resuscitation system may include a sensor for measuring characteristics (quality) of the ventilation, such as ventilation rate and volume. The resuscitation system may also comprise feedback devices, such as speaker or display, to give feedback to the operator on the performed ventilations, position stability of the chest compression device, time left of battery, stiffness changes in the patient's chest, or other aspects regarding the ventilation system or the patient.

The resuscitation system can comprise power supply devices comprising energy storage devices or devices for connection to power sources in an ambulance, in a hospital or in an external power storage device.

The may further include a user interface (not shown) for providing information regarding the resuscitation. The user interface may provide information related to the service level of the system, remaining power, defibrillator status, ventilation status or other information which would be useful for the operator during or after the resuscitation.

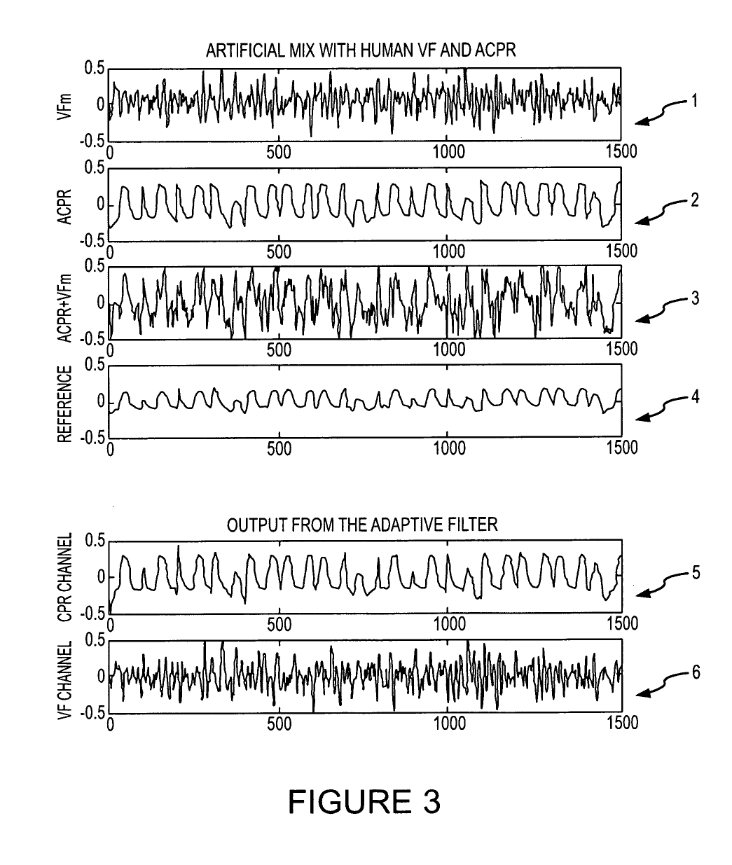

FIG. 3 shows an example of ventricular fibrillation (VF) signals and signals caused by CPR. Curve 1 shows a VF curve from a person without signal disturbance. Curve 2 shows signal disturbances during CPR measured on a pig. Curve 3 shows the sum of signals 1 and 2 and simulates/represents a VF signal disturbed by CPR. Curve 4 shows a reference signal, which is provided by position and impedance measurements. These measurements may be used to estimate the disturbance signal from CPR, an example of such an estimate is shown as curve 5. Subtracting the estimated disturbance from the disturbed VF signal (i.e. curve 3), provides an estimated “undisturbed” curve 6 which may be used for deciding when to activate the defibrillator.

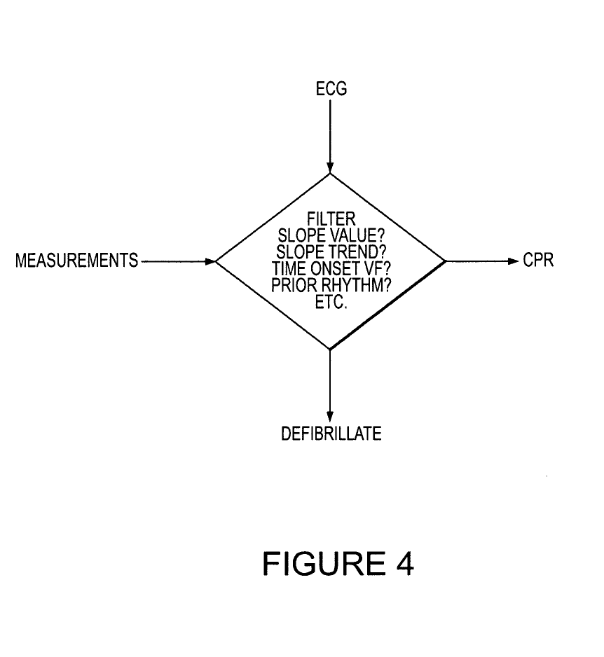

An exemplary decision model is shown in FIG. 4. The signals from ECG and CPR sensors are used to provide an estimated ECG curve, which is analysed with respect to for example slope characteristics, VF detection, prior rhythm, etc. The slope value can give an indication of the effect of defibrillation, a high value indicates good effect, while a low value indicates poor effect. A positive trend of the slope may indicate that ongoing CPR should be continued. The estimated ECG curve may be provided continuously or intermittent. The analysis of the estimated ECG curve leads to a decision on whether CPR should be performed or a defibrillator shock should be activated.

From the foregoing it will be appreciated that, although specific embodiments of the invention have been described herein for purposes of illustration, various modifications may be made without deviating from the spirit and scope of the invention. Accordingly, the invention is not limited except as by the appended claims.

What is claimed is:

1. A resuscitation system, comprising:
a chest compression device structured to repeatedly compress the chest of a patient and thereafter cause or allow the chest to expand,
a defibrillator operable to apply electric impulses to a heart,
a measuring device operable to measure at least one characteristic of the resuscitation process, and
a signal processor connected to the chest compression device and the defibrillator, the signal processor being operable to control operation of at least one of the chest compression device and the defibrillator.

2. The resuscitation system of claim 1 wherein the signal processor is operable to control the operation of at least one of the chest compression device and the defibrillator based on the at least one measured characteristics of the resuscitation process.

3. The resuscitation system of claim 1 wherein the signal processor is operable to control the operation of at least one of the chest compression device and the defibrillator based on a predetermined characteristic of the resuscitation process.

4. The resuscitation system of claim 1 wherein the signal processor is operable to control the operation of at least one of the chest compression device and the defibrillator based on a comparison between the at least one measured characteristics of the resuscitation process and at least one predetermined characteristic of the resuscitation process.

5. The resuscitation system of claim 1 wherein the signal processor comprises a filter for attenuating signal noise caused by chest compression in an ECG and/or impedance signal.

6. The resuscitation system of claim 1 wherein the measuring device comprises at least one of a force sensor and a depth sensor.

7. The resuscitation system of claim 1 wherein the measuring device comprises at least one of a blood flow sensor and a blood pressure sensor.

8. The resuscitation system of claim 1 wherein the measuring device comprises a ventilation sensor.

9. The resuscitation system of claim 1 further comprising a ventilation device.

10. The resuscitation system of claim 1 wherein the measuring device or the defibrillator device comprises an ECG device.

11. The resuscitation system of claim 1, further comprising a power supply device.

12. The resuscitation system of claim 1 wherein the power supply device comprises an energy storage device.

13. The resuscitation system of claim 1 wherein the power supply device comprises a device for connection to an external power storage device including a power source in an ambulance or hospital.

14. The resuscitation system of claim 1, further comprising a data storing device connected to the measuring device, the data storage device being operable to store values indicative of the at least one characteristic of resuscitation measured by the measuring device.

15. The resuscitation system of claim 1, further comprising a user interface operable to provide information regarding the resuscitation.

16. The resuscitation system of claim 1 wherein the defibrillator device comprises an automatic external defibrillator (AED).

17. The resuscitation system of claim 1, further comprising a support connected to the chest compression device.
